(19) United States
(12) Patent Application Publication
(10) Pub. No.: US 2007/0172466 A1
(43) Pub. Date: Jul. 26, 2007

(54) METHOD OF REPLENISHING CELLS DAMAGED BY TREATMENT FOR CANCER

(76) Inventor: Donnie RUDD, Sugar Land, TX (US)

Correspondence Address: LADAS & PARRY LLP, 224 SOUTH MICHIGAN AVENUE, SUITE 1600, CHICAGO, IL, 60604, US

(21) Appl. No.: 11/614,345

(22) Filed: Dec. 21, 2006

Related U.S. Application Data
(63) Continuation-in-part of application No. 10/233,344, filed on Sep. 3, 2002.

(51) Int. Cl. A61K 48/00; A61K 35/14; C12N 5/08

(52) U.S. Cl. 424/93.21; 424/93.7; 435/372

(57) ABSTRACT

A method of replenishing cells damaged by treatment for cancer comprising removing a sample of peripheral blood containing CD34+ pluripotent adult stem cells from a primate mammal, controllably expanding the cells without differentiation at a rate which produces an expansion factor of at least seven times within seven days while maintaining their three-dimensional geometry and their cell-to-cell support and cell-to-cell geometry, removing any toxic materials from the expanded undifferentiated CD34+ pluripotent adult stem cells, and reintroducing the CD34+ pluripotent adult stem cells into the primate mammal within a time period sufficient to prevent the primate mammal from suffering decreased mobility due to loss of hematopoietic or other cells from a treatment for cancer.

This is a Continuation in Part Application, the parent application being Ser. No. 10/233,344 filed on Sep. 3, 2002.

### BACKGROUND OF THE INVENTION

The present invention relates to a method of replenishing cells damaged by treatment for cancer.

One of the worst side effects of the use of chemotherapy in the treatment for cancer is the loss of energy by the patient due to loss of red blood cells. In the process of destroying cancer cells, chemotherapy often causes damage to other rapidly dividing cells, such as the bone marrow cells. Bone marrow is responsible for producing red blood cells, white blood cells, and platelets. The reduced activity of the bone marrow is named myelosuppression. Chemotherapy and radiation can depress the number of red blood cells to a low level and eventually produce tiredness, lack of energy, and anemia. Myelosuppression is the dose-limiting toxicity of most highly effective chemotherapeutic agents. In recent years this limitation has been overcome through the use of SC transplantation (SCT). In fact, SCT performed after high-dose chemotherapy allows further escalation of dose intensity, thus increasing survival in many patients with advanced malignant diseases. Nevertheless, most patients treated with SCT experience prolonged neutropenia and thrombocytopenia resulting in increased morbidity and mortality.

Labeled by some as cancer's number one side effect, fatigue is part of the illness of 72% to 95% of patients with cancer. Chronic or acute—some describe it as “hitting a wall”—the fatigue experienced by patients with cancer differs from that of healthy people. It is debilitating and depressing, it interferes with normal activities, and it is a barrier to a person's enjoyment of life. The National Cancer Institute describes fatigue's social implications as potentially “profound.”

Fatigue, long discounted, has become more prominent because therapies have become more aggressive and exacerbated it and because health professionals have acknowledged it as a dose-limiting toxicity of therapy and as a quantifiable and treatable side effect. It is emerging as a serious topic of research, which encompasses biochemical, pathophysiologic, psychologic, and behavioral variables. Unfortunately, while medical science has been making steady progress in treating cancer itself, cancer related fatigue is frequently over-looked, under-recognized and under-treated. Aside from the discomfort of feeling exhausted, fatigue can pose a number of obstacles to coping with cancer and reaping the full benefits of available treatments. Fatigue can significantly interfere with a patient's quality of life and may limit the number of chemotherapy cycles that could be administered, which may limit the effectiveness of treatment altogether.

In the past, the preferred treatment for fatigue associated with cancer treatment has been the administration of medication such as epoetin alfa (Procrit), or when the condition becomes severe, a transfusion of red blood cells.

None of the currently available medications, such as epoetin alfa, provide full relief from fatigue due to chemotherapy. While they assist in reducing some of the problems and providing some relief, the medications also have side effects, which create a new series of problems for the patient. Likewise, a transfusion of red blood cells is generally administered only after the patient has suffered the worst effects of the fatigue.

It can therefore be seen that a need exists to minimize the fatigue associated with chemotherapy or radiation for cancer in order to provide a better quality of life for patients undergoing treatment for cancer.

### SUMMARY OF THE INVENTION

The present invention relates to a method of replenishing cells damaged by treatment for cancer comprising removing a sample of peripheral blood cells from a primate mammal, determining the level of CD34+ pluripotent adult stem cells in the peripheral blood, controllably expanding the CD34+ pluripotent adult stem cells by increasing the number of CD34+ pluripotent adult stem cells by a factor of at least seven times within seven days without differentiating the cells and without adding a dipeptidyl peptidase inhibitor, while at the same time, maintaining the CD34+ pluripotent adult stem cells three-dimensional geometry and their cell-to-cell support and cell-to-cell geometry, removing any toxic materials from the undifferentiated expanded CD34+ pluripotent adult stem cells and reintroducing the undifferentiated expanded CD34+ pluripotent adult stem cells into the primate mammal within a time period sufficient to prevent the primate mammal from suffering decreased mobility due to loss of hematopoietic or other cells from a treatment for cancer.

These and still other objects and advantages of the present invention will be apparent from the description of the preferred embodiments that follow. However, the claims should be looked to in order to judge the full scope of the invention.

### DETAILED DESCRIPTION OF THE INVENTION

This invention may be more fully described by the preferred embodiment as hereinafter described.

In one preferred embodiment of the present invention, a sample of CD34+ pluripotent adult stem cells are removed from the peripheral blood of a cancer patient prior to chemotherapy treatment. The CD34+ adult stem cells are placed in a bioreactor, preferably rotatable. The bioreactor is rotated at a speed that provides for suspension of the CD34+ pluripotent adult stem cells to maintain their three-dimensional geometry and their cell-to-cell support and geometry, During the CD34+ pluripotent adult stem cell expansion in the bioreactor, the cells are fed nutrients and toxic materials are removed. The CD34+ pluripotent adult stem cells are expanded by a factor of at least seven times within seven days. The cells of the present invention are expanded without fortifying the culture mix with any additional dipeptidyl peptidase (“DPIV”) inhibitor and without differentiation of the cells. By the term “fortifying the culture mix” it is intended that no additional DPIV inhibitor is added to the culture mix, over and above that already present, if any, in the sample of cells removed from the patient. Furthermore, at no time during the expansion step is it intended that a DPIV inhibitor be added. The patient is then administered chemotherapy. The undifferentiated expanded CD34+ pluripotent adult stem cells are then reintroduced to the patient within a time period sufficient to prevent the primate mammal from suffering decreased mobility due to loss of hematopoietic or other cells from the chemotherapy.

In still another embodiment of this invention, peripheral blood (PB) cells are obtained from normal stem cell (SC) donors. In brief, mononuclear cells (MNCs) are obtained from the first apheresis product collected from SC donors. Prior to apheresis, each donor is treated with G-CSF 6:g/kg every 12 hr over 3 days and then once on day 4. MNCs are collected by subjecting each donor's total blood volume to 3 rounds of continuous-flow leukapheresis through a Cobe Spectra cell separator.

In operation, a culture mix is created by suspending MNCs (0.75 ×10⁶ cells/ml) in Iscove's modified Dulbecco's medium (IMDM) (GIBCO, Grand Island, N.Y.) preferably supplemented with 5% human albumin (HA) or more preferably 20% human plasma, 100 ng/ml recombinant human G-CSF (Amgen, Inc., Thousand Oaks, Calif.), and 100 ng/ml recombinant human stem cell factor (SCF) (Amgen). The culture mix is placed in a rotatable bioreactor and then placed in a humidified incubator at 37EC under an atmosphere of 5% CO₂. By “placed in” it is intended that either the cells can be added to the bioreactor after the medium and other preferred ingredients are already therein, or the cells can be mixed with the medium and other preferred ingredients before adding the culture mix to the bioreactor. The cells in the culture mix are preferably inspected daily. On days 2, 5, 6, and 7 a sample of cells is removed from the bioreactor and counted using the trypan-blue exclusion test.

Hematopoietic colony-forming cells may preferably be assayed using a modification of a previously described assay. In brief, 10⁵ cells/ml (MNCs) may preferably be mixed with 0.8% methylcellulose with IMDM, 30% FCS, 1.0 U/ml erythropoietin (Amgen), 50 ng/ml recombinant human GM-CSF (Immunex Corp., Seattle, Wash.), and 50 ng/ml SCF (Amgen). All cultures are preferably evaluated after 7 days for the number of burst-forming unit-erythroid (BFU-E) colonies (defined as aggregates of more than 500 hemoglobinized cells or 3 or more erythroid subcolonies), for the number of colony-forming units granulocyte-macrophage (CFU-GM) colonies of granulocytic or monocyte-macrophage cells or both, and for the number of CFU-granulocyte-erythroid-macrophage-megakaryocyte (CFU-GEMM) containing all elements. Samples of cells are removed from the bioreactor and analyzed for cellular composition.

Lymphocytes are preferably analyzed by 2-color staining using the following antibody combinations: CD56+CD16-PE/CD3-FITC, CD3-PE/CD4-FITC, CD3-PE/CD8-FITC, CD19-PE. Controls include IgG1-PE/lgG1-FITC for isotype and CD14-PE/CD45-FITC for gating. CD34+ adult stem cells are preferably analyzed by 3-color staining with the fluorochromes PerCP/PE/FITC using the following antibody combinations: CD45/CD90/CD34, CD45/CD34/CD38, CD45/CD34/CD33, and CD45/CD34/CD15. CD45/IgG1/Ig1 is used as a control. Preferably, 10⁶ cells from each donor are incubated with 10:1 of antibodies at 2-8EC for 15 minutes in the dark and then washed twice in phosphate-buffered saline. Then the cells are preferably resuspended, fixed with 1% formaldehyde, and analyzed on a FACScan flow cytometer (Becton-Dickinson) equipped with CELLQuest software (Becton-Dickinson). For analyses of lymphocytes, preferably 10,000 cells are acquired from each tube, and then gated on the basis of the forward and right angle light scatter patterns. The cutoff point is visually set at a level above background positivity exhibited by isotype controls. For analyses of progenitor cells, 75,000 cells from each tube is acquired and then sequentially gated.

Incubation of donors' PB cells in my tissue culture system significantly increases the numbers of hematopoietic colony-forming cells. An increase in the numbers of CFU-GM (up to approximately 7-fold) and CFU-GEMM (up to approximately 9-fold) colony-forming cells is expected by day 7 with no clear plateau.

Incubation of MNCs from normal donors is expected to significantly increase the numbers of CD34+ pluripotent adult stem cells. Preferably, the average number of CD34+ pluripotent adult stem cells increases 10-fold by day 6 of culture. Preferably, the relative number of CD34+ pluripotent adult stem cells co-expressing the myeloid-lineage markers CD15 and CD33 also increases significantly by days 5 and 6.

Preferably, within one week of the chemotherapy treatment, the undifferentiated expanded CD34+ pluripotent adult stem cells are reintroduced into the body. Without being bound by theory, reintroducing the undifferentiated expanded CD34+ pluripotent adult stem cells should allow the body to maintain a sufficient level of replenished cells to overcome the fatigue caused by the chemotherapy.

Even though the preferred embodiment of this invention is described above, it will be appreciated by those skilled in the art that other modifications can be made within the scope of this invention.

I claim:

1. A method of replenishing cells damaged by treatment for cancer comprising:
removing a sample of peripheral blood cells from a primate mammal;
determining the level of CD34+ pluripotent adult stem cells in the peripheral blood cell sample;
controllably expanding without differentiating the CD34+ pluripotent adult stem cells from the peripheral blood cell sample by increasing the number of CD34+ pluripotent adult stem cells from the peripheral blood cell sample by a factor of at least seven times while maintaining their three-dimensional geometry and their cell-to-cell support and cell-to-cell geometry;
removing any toxic materials from the expanded undifferentiated CD34+ pluripotent adult stem cells; and
a reintroducing the expanded undifferentiated CD34+ pluripotent adult stem cells into the primate mammal within a time period sufficient to prevent the primate mammal from suffering decreased mobility due to loss of hematopoietic or other cells from a treatment for cancer.

2. A method as in claim 1 wherein the primate mammal is a human.

3. A method as in claim 1 wherein the expanded undifferentiated CD34+ pluripotent adult stem cells are reintroduced into the primate mammal to prevent the primate mammal from suffering decreased mobility due to loss of hematopoietic or other cells from a treatment for cancer that comprises chemotherapy.

4. A method as in claim 1 wherein the expanded undifferentiated CD34+ pluripotent adult stem cells are reintroduced into the primate mammal to prevent the primate mammal from suffering decreased mobility due to loss of hematopoietic or other cells from a treatment for cancer that comprises radiation.

5. A method as in claim 1 wherein the reintroduction of the expanded undifferentiated CD34+ pluripotent adult stem cells is accomplished within 1 month of the treatment for cancer.

6. A method as in claim 1 wherein the reintroduction of the expanded undifferentiated CD34+ pluripotent adult stem cells is accomplished within 1 week of the treatment for cancer.
